(19) United States
(12) Patent Application Publication
(10) Pub. No.: US 2003/0211531 A1
(43) Pub. Date: Nov. 13, 2003

(54) Methods for discovering tumor biomarkers and diagnosing tumors

(75) Inventors: Garret M. Hampton, San Diego, CA (US); John B. Welsh, San Diego, CA (US)

Correspondence Address: TIMOTHY L. SMITH, GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton HM LX (BM)

(21) Appl. No.: 10/429,221

(22) Filed: May 1, 2003

Related U.S. Application Data
(60) Provisional application No. 60/442,853, filed on Jan. 24, 2003.
(60) Provisional application No. 60/377,402, filed on May 1, 2002.

(51) Int. Cl. C12Q 1/68; G06F 19/00; G01N 33/48; G01N 33/50

(52) U.S. Cl. 435/6; 702/20

(57) ABSTRACT

This invention provides methods for discovery of biomarkers that are useful for diagnosing, prognosticating, and monitoring the treatment efficacy, of various cancers. The invention also provides methods for diagnosing various forms of cancers using biomarkers identified in accordance with the invention.

### CROSS-REFERENCE TO RELATED APPLICATIONS

This application claims the benefit of priority to U.S. Provisional Patent Application Serial No. 60/442,853 (filed Jan. 24, 2003) and No. 60/377,402 (filed May 1, 2002). The full disclosures of these applications are incorporated herein by reference in their entirety and for all purposes.

### FIELD OF THE INVENTION

The present invention relates generally to genes useful as diagnostic markers and/or targets for therapeutic intervention in cancers. More particularly, the present invention concerns the identification of genes that encode secreted proteins and are differentially expressed in malignant and normal tissues. Methods are provided for the diagnosis, prognosis and treatment of various cancers based upon these genes.

### BACKGROUND OF THE INVENTION

Cancer is a leading cause of death in the United States, causing one in four deaths, which is second only to heart disease. More than half a million people die of cancer each year in the United States. Four cancer sites, the lung, prostate, breast and colon, account for 56 percent of all new cancer cases and are the leading causes of cancer deaths for every racial and ethnic group, according to the Annual Report to the Nation on the Status of Cancer, 1973-1998 (Howe et al. (2001) J. Nat'l. Cancer Institute 93: 824-842). The early stages of these and other types of cancer are often curable by, for example, surgery, radiation therapy or chemotherapy. Accordingly, early diagnosis of cancer is critical for effective treatment.

All too often, patients die because their cancer is not diagnosed until after the window for successful intervention has closed. The problem is exacerbated by serious shortcomings in previously existing methods for cancer diagnosis. First, in about four percent of all patients diagnosed with cancer, the observed tumor is due to metastasis and the primary tumor origin is undetermined. The inability to identify the site of the primary tumor complicates diagnosis and treatment (Hillen, Postgrad. Med. J. 76: 690-693 (2000)). Even for patients in which the primary tumor is known, existing diagnostic methods are less than optimal. For prostate cancer, as an example, detection efforts based on prostate-specific antigen (PSA) screening as described, e.g., in Catalona et al., JAMA 270: 948-954 (1993), have led to the identification of thousands of men with localized disease. Although serum PSA is widely recognized as the best prostate tumor marker currently available as described, e.g., in Brawer, Semin. Surg. Oncol., 18: 3-9 (2000), screening programs utilizing PSA alone or in combination with digital rectal examination have failed to improve the survival rate of men with prostate cancer.

Several disadvantages attend the use of PSA as a diagnostic marker. First, while PSA is specific for prostate tissue, it is produced by normal as well as malignant prostate tissue, and quantification of PSA expression in a fragment of prostate tissue does not unambiguously classify that tissue with respect to malignancy or malignant potential. Second, not every prostate tumor secretes PSA. Third, while high PSA serum levels are an effective indicator of prostate cancer, modestly elevated levels, e.g., between 4 and 10 ng/mL are seen in men with obstructive or inflammatory uropathies, lowering the specificity of PSA as a cancer marker as described, e.g., in Brawer et al., Am. J Clin. Pathol., Vol. 92, pp. 760-764 (1989). Other biomarkers such as glandular kallikrein 2 (hK2) and prostate specific transglutaminase (pTGase), have been proposed as adjuncts to PSA to increase diagnostic specificity as described, e.g., in Nam et al., J Clin. Oncol., Vol. 18, pp. 1036-1042 (2000), and reduce the number of men subjected to unnecessary biopsy, but the usefulness of these markers is still under investigation.

Early detection of cancer via serologic immunoassay represents a critical goal toward significantly improving the diagnosis and prognosis of cancer patients. The present invention fulfills this and other needs.

### SUMMARY OF THE INVENTION

The invention provides methods for identifying a biomarker that is diagnostic for the presence of a cancer in a mammal. The methods involve: (a) analyzing one or more polynucleotide sequences using an algorithm that determines whether the polynucleotide sequence is predicted to encode a polypeptide that is secreted from a cell in which the polypeptide is expressed; and (b) determining whether an mRNA that corresponds to one or more of the polynucleotide sequences that are predicted to encode secreted polypeptides is differentially expressed in one or more types of cancer cells compared to non-cancer cells. An mRNA that is differentially expressed in cancer cells compared to non-cancer cells, or a polypeptide encoded by the differentially expressed mRNA, is useful as a biomarker that is diagnostic for the presence of the cancer in a mammal.

Also provided by the invention are methods of diagnosing a cancer in a mammal. These methods involve detecting in a sample obtained from the mammal an increase in the level of a biomarker, wherein the biomarker was identified using a method that comprises: (a) analyzing one or more polynucleotide sequences using an algorithm that determines whether the polynucleotide sequence is predicted to encode a polypeptide that is secreted from a cell in which the polypeptide is expressed; and (b) determining whether an mRNA that corresponds to one or more of the polynucleotide sequences that are predicted to encode secreted polypeptides is differentially expressed in one or more types of cancer cells compared to non-cancer cells. An mRNA that is differentially expressed in cancer cells compared to non-cancer cells, or a polypeptide encoded by the differentially expressed mRNA, is useful as a biomarker that is diagnostic for the presence of the cancer in a mammal.

The invention also provides methods for monitoring the efficacy of a cancer treatment in a mammal. These methods involve detecting an increase or decrease in the level of a biomarker that is diagnostic for the presence of the cancer in a mammal in a plurality of samples obtained from the mammal at different times, wherein the biomarker was identified using a method that comprises: (a) analyzing one or more polynucleotide sequences using an algorithm that determines whether the polynucleotide sequence is predicted to encode a polypeptide that is secreted from a cell in which the polypeptide is expressed; and (b) determining whether an mRNA that corresponds to one or more of the polynucleotide sequences that are predicted to encode secreted polypeptides is differentially expressed in one or more types of cancer cells compared to non-cancer cells. An mRNA that is differentially expressed in cancer cells compared to non-cancer cells, or a polypeptide encoded by the differentially expressed mRNA, is useful as a biomarker that is diagnostic for the presence of the cancer in a mammal.

Further provided by the invention are methods for diagnosing or identifying a predisposition to the development of a cancer, using the specific cancer biomarkers identified by the present inventors as shown in Table 1. These methods entail (a) obtaining a biological sample (e.g., serum) from a subject (e.g., a mammal) suspected to have or be predisposed to develop the cancer; and (b) detecting in the biological sample an abnormal level of at least one secreted biomarker for that cancer. For example, the secreted biomarkers for diagnosing prostate cancer can include relaxin 1 (H1), neuropeptide Y, MIC-1, pancreatic thread protein-like (rat), prostate-specific membrane antigen, prostate-specific membrane antigen, prostate-specific membrane antigen, and single-minded homolog 2 (Drosophila).

### BRIEF DESCRIPTION OF THE DRAWINGS

FIG. 1 shows a schematic of a strategy for identifying genes that encode secreted proteins that are differentially expressed in cancer tissues.

FIGS. 2A-2B show that expression of candidate secreted biomarkers are elevated in multiple cancer-types. Thirty-two genes encoding secreted proteins selected by annotation- and sequence-based analyses had significant overexpression in at least one tumor-normal counterpart tissue pair (>3-fold), and significant overexpression in tumors compared to any other normal tissue (>2-fold). BR—breast (ER+, ER−); CO—colorectal, GA—gastric/esophagus adenocarcinoma; KI—kidney, LI—liver; LUA—adenocarcinoma of the lung; LUS—squamous carcinoma of the lung; LUO—lung “other”—small cell lung carcinomas, large cell undifferentiated carcinomas of the lung; OV—ovary; PA—pancreas; PR—prostate. Gene symbols are depicted to the right of the figure (FIG. 2A). An expanded view of genes preferentially upregulated in carcinomas of the prostate is shown in FIG. 2B. Numbers of tissue samples from each counterpart site are given in parentheses; “geneatlas” tissues are, Te—testis; Th—thyroid; Ut—uterus; SG—salivary gland; Tr—trachea; AG—adrenal gland; He—heart; Pi—pituitary gland; Sp—spinal cord; CC—cerebral cortex; Nor. Pr—normal prostate Transcript levels were normalized in Cluster and visualized in TreeView (Eisen et al., Proc Natl Acad Sci U S A 95, 14863-8, 1998).

FIGS. 3A-3B show validation of microarray gene expression by RT-PCR, IHC and ELISA. FIG. 3A shows RNAs expressed from multiple different human tissues (labeled above the RT-PCR panel), three normal, and six primary prostate carcinomas were reverse transcribed and amplified under standard conditions using primers directed toward relaxin-1. The primary microarray data is shown at top (hybridization intensity on the Y-axis; samples on the X-axis), and a representative PCR is shown at bottom. Primers specific for 18S were used to control for the amount of amplified cDNA. FIG. 3B shows IHC performed with an anti-NPY antibody on whole tissue sections. Primary microarray data is shown at top, with examples of IHC staining in normal, microarray-positive and microarray-negative prostate cancers.

FIGS. 4A-4C show validation of increases in the levels of candidate diagnostic proteins. Antibodies specific to candidate secreted proteins, NPY (FIG. 4A), MUC-2 (FIG. 4B) and Maspin (FIG. 4C), were used to stain tissue microarrays containing 36 normal epithelial tissues and 229 carcinomas. The relative levels of expression for each gene are depicted at the top of each figure, with groups of tissues, and specific tissues identified. Gene expression levels were output in TreeView. Examples of IHC staining are shown at the bottom of each figure, highlighting both negative and positive cancers for each protein.

FIGS. 5A-5B show that upregulation of maspin expression correlates with estrogen receptor status in breast carcinomas. FIG. 5A shows expression of maspin monitored by two independent probe-sets of the Affymetrix U95a GeneChip (PS 1 and PS2). FIG. 5B shows comparison of microarray data and IHC on tissue microarrays in normal, ER+ and ER− tumors using an anti-maspin antibody. Note the intense staining on ER− tumors compared with normal ductal breast tissue

FIG. 6 shows expression of candidate lung cancer markers in an expanded set of normal and tumor lung tissue samples. The expression levels of lung cancer candidate genes (derived from each group of candidates—GO annotation and sequence-positive; GO annotation-positive only and sequence-positive only; Table 1) was determined in normal lung, lung adenocarcinomas, small cell undifferentiated carcinomas, squamous carcinomas and carcinoids. Data from the study (available at http://research.dfci.harvard.edu/meyersonlab/lungca/data.html) were downloaded and output in TreeView. Note the high levels of expression of GRP in carcinoids and SCLC, and the near-uniform overexpression of maspin in squamous carcinomas.

### DETAILED DESCRIPTION

The invention provides a global approach to the discovery of secreted, cancer-specific biomarkers. The methods involve identifying nucleic acids that encode proteins that are likely to be secreted from cells in which the proteins are expressed, and from that set of secreted protein-encoding nucleic acids, identifying those that exhibit differential expression in cancer cells compared to non-cancer cells. By focusing the approach on secreted proteins, one can identify biomarkers that can be detected in samples obtained from a human or other mammal, thus facilitating the diagnosis of cancer in the mammal. In preferred embodiments, the biomarker polypeptides are detectable in the blood, serum, or other biological sample that is readily obtainable from the mammal.

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Although any methods and materials similar or equivalent to those described herein can be used in the practice or testing of the present invention, the preferred methods and materials are described.

Nucleic acids that encode secreted proteins can be identified using methods known to those of skill in the art. For example, secreted proteins can be identified by virtue of an annotation associated with a nucleotide or amino acid sequence that is present, for example, in a database. One example of suitable sources of such annotated sequences are the databases provided by the Genome Ontology Consortium (http://www.genomeontology.org). One can search the database for sequences that are annotated as encoding proteins that are found in cellular locations that are indicative of secretion.

Alternatively or additionally, use of annotations to identify polynucleotides that are associated with secreted proteins, one can use one or more of the computer-implemented algorithms that are useful for determining whether a polypeptide is secreted. For example, such algorithms can determine whether an amino acid sequence includes, for example, a transmembrane domain. A suitable algorithm for this analysis is the “Tmap” program (Persson et al., J. Mol Biol, 237:182-192, 1994). This program is available on the internet at, for example, http://www.mbb.ki.se/tmap/. Secreted polypeptides can also be identified using an algorithm that identifies amino acid sequences within the polypeptide that comprise signal peptides, and/or that recognize cleavage sites for signal polypeptides. An example of software that conducts this type of analysis is “Sigcleave” (von Heijne et al., Nucl. Acids Res., 14:4683-4690, 1986). Sigcleave estimates the likelihood of an authentic signal peptide cleavage site in arbitrary amino acid sequence data. Sigcleave is available on the internet at, for example, http://bioweb.pasteur.fr/seqanal/interfaces/sigcleave.html.

The set of polynucleotides that encode proteins that are predicted to be secreted from a cell are then subjected to expression analysis in tumor cells and non-tumor cells to identify those that exhibit differential expression in tumor cells compared to non-tumor cells. These secreted, differentially expressed polypeptides are suitable for use as biomarkers for the cancers in which the polypeptides are overexpressed.

The level of expression of at least one of the genes that encode secreted polypeptides in the samples obtained from the subject and disease-free subject can be detected by measuring either the level of mRNA corresponding to the gene, the protein encoded by the gene or a fragment of the protein. In the methods of the invention, the level of expression of one of the disclosed genes in a cancer tissue preferably differs from the level of expression of the gene in a non-cancer tissue by a statistically significant amount. In presently preferred embodiments, at least about a 2-fold difference in expression levels is observed. In some embodiments, the expression levels of a gene differ by at least about 3-, 5-, 10- or 100-fold or more in the cancer tissue compared to the non-cancer tissue.

RNA can be isolated from the samples by methods well known to those skilled in the art as described, e.g., in Ausubel et al., Current Protocols in Molecular Biology, Vol. 1, pp. 4.1.1-4.2.9 and 4.5.1-4.5.3, John Wiley & Sons, Inc. (1996). Methods for detecting the level of expression of mRNA are well known in the art and include, but are not limited to, northern blotting, reverse transcription PCR, real time quantitative PCR and other hybridization methods. A particularly useful method for detecting the level of mRNA transcripts obtained from a plurality of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can be compared with the gene expression profile derived from the sample obtained from the cancer-free subject to determine whether the genes are over-expressed in the sample from the subject relative to the genes in the sample obtained from the disease-free subject, and thereby determine whether the subject has or is at risk of developing a cancerous disorder (e.g., prostate cancer or colon cancer).

The oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides can be bound, either directly or indirectly, either covalently or non-covalently, can be used. A particularly preferred solid substrate is a high density array or DNA chip (e.g., the U95a GeneChip™ from Affymetrix Inc., Santa Clara, Calif.). These high density arrays contain a particular oligonucleotide probe in a pre-selected location on the array. Each pre-selected location can contain more than one molecule of the particular probe. Because the oligonucleotides are at specified locations on the substrate, the hybridization patterns and intensities (which together result in a unique expression profile or pattern) can be interpreted in terms of expression levels of particular genes.

The oligonucleotide probes are preferably of sufficient length to specifically hybridize only to complementary transcripts of the above identified gene(s) of interest. As used herein, the term “oligonucleotide” refers to a single-stranded nucleic acid. Generally the oligonucleotides probes will be at least 16 to 20 nucleotides in length, although in some cases longer probes of at least 20 to 25 nucleotides will be desirable.

The oligonucleotide probes can be labeled with one or more labeling moieties to permit detection of the hybridized probe/target polynucleotide complexes. Labeling moieties can include compositions that can be detected by spectroscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled binding proteins, heavy metal atoms, spectroscopic markers such as fluorescent markers and dyes, linked enzymes, mass spectrometry tags, and magnetic labels.

Oligonucleotide probe arrays for expression monitoring can be prepared and used according to techniques which are well known to those skilled in the art as described, e.g., in Lockhart et al., Nature Biotechnology, Vol. 14, pp. 1675-1680 (1996); McGall et al., Proc. Natl. Acad. Sci. USA, Vol. 93, pp. 13555-13460 (1996); and U.S. Pat. No. 6,040,138.

Expression of the protein encoded by the gene(s) or a fragment of the protein can be detected by a probe which is detectably labeled, or which can be subsequently labeled. Generally, the probe is an antibody which recognizes the expressed protein. In some embodiments, expression of a protein in multiple tissues can be analyzed with tissue microarrays (TMAs) and immunohistochemistry. Tissue microarrays can be constructed according to methods routinely practiced in the art. For example, microarrays containing multiple tissue samples can be prepared using a Tissue Microarrayer (Beecher Instruments, Silver Spring, Md.) with, e.g., zinc formalin-fixed, paraffin-embedded specimens. Each microarray can contain one core of each neoplasm whose transcripts are profiled in the analysis. Thus, a tissue microarray can comprise a set of tissues from different carcinomas, as well as cores of selected normal tissues (see the Examples below).

As used herein, the term antibody includes, but is not limited to, polyclonal antibodies, monoclonal antibodies, humanized or chimeric antibodies and biologically functional antibody fragments, which are those fragments sufficient for binding of the antibody fragment to the protein or a fragment of the protein.

Antibodies used in IHC (immunohistochemistry) analysis of the TMAs can be generated using methods well known and routinely practiced in the art. Some antibodies employed to practice the present invention can also be obtained commercially, e.g., monoclonal anti-MUC-2 (BioGenex, San Ramon, Calif.); monoclonal anti-maspin (Novocastra Laboratories, Newcastle upon Tyne, UK); and rabbit polyclonal anti-neuropeptide Y (Research Diagnostics, Inc, Flanders N.J.).

For the production of antibodies to a protein encoded by one of the disclosed genes or to a fragment of the protein, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Liss, Inc., pp. 77-96 (1985)). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide.

Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity.

The extent to which the known proteins are expressed in the sample is then determined by immunoassay methods which utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof, e.g., PLAB or the catalytic domain of hepsin, which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

In another aspect, the present invention provides methods for diagnosing various forms of cancers or a predisposition to develop any of the cancers. The methods comprise detecting at least one (e.g., 1, 2, 3, 4, 5, or more) differentially expressed cancer-specific biomarkers for a given cancer that have been identified in accordance with the present invention (e.g., see Table 1). Typically, a diagnostic test works by comparing a measured level of at least one biomarker (e.g., MIC-1) in a subject (e.g., a mammal) with a baseline level determined in a control population of subjects unaffected by cancer. In some cancers, abnormal expression of a biomarker is limited to a specific tissue type (e.g., breast tissue for breast cancer). In such cases, the baseline level of the biomarker for comparison can also be an expression level of the biomarker in control tissues where the cancer is not present.

If the measured level does not differ significantly from baseline levels in a control population (or control tissues), the outcome of the diagnostic test is considered negative. On the other hand, if there is a significant departure between the measured level in a subject and baseline levels in unaffected subjects (or control tissues), it signals a positive outcome of the diagnostic test, and the subject is considered to have abnormal presence or an abnormal level of that biomarker. In general the departure is an increase in expression levels of the biomarkers. However, for some biomarkers of certain cancers, abnormality can also be a decreased expression level.

As noted above, in preferred embodiments, a departure from baseline levels is statistically significant if at least a 2-fold difference in expression levels is observed. However, depending on the specific case (the cancer and the biomarker), a departure with less than a 2-fold difference in the expression levels can still be considered significant, if the measured value falls outside the range typically observed in unaffected subjects due to inherent variation between subjects and experimental error. For example, in some methods, a departure can be considered significant if a measured level does not fall within the mean plus one standard deviation of levels in a control population. Thus, a significant departure may occur if the difference between the measured level and baseline levels is at least 20%, 30%, 40%, 50%, 60%, 70%, 80%, or 90%. The extent of departure between a measured value and a baseline value in a control population also provides an indicator of the probable accuracy of the diagnosis, and/or of the severity of the disease being suffered by the subject.

Other than measuring and comparing expression level of individual biomarkers, the methods for diagnosing cancers can entail obtaining from a subject an expression profile of biomarkers for a given cancer, and comparing the gene expression profile to at least one expression profile from subjects known to have the cancer. The profile can contain expression levels (e.g., in the serum) of at least one (e.g., 1, 2, 3, 4, 5, or more) biomarkers for that cancer. Methods of obtaining expression profiles and their uses in disease diagnosis are well known in the art. For example, methods of the present invention can be practiced using the specific biomarkers identified by the present inventors with techniques described in, e.g., U.S. Pat. No. 6,365,352 or WO0111082.

For the diagnostic methods, a preferred biological sample for measuring levels of the secreted cancer biomarkers is serum. Other tissue samples from blood, e.g., whole blood and plasma, may also be used to measure levels of the secreted biomarkers in a subject and the control population.

Other than blood related biological samples, other samples may also be employed for measuring expression levels of the cancer biomarkers. These include, e.g., samples obtained from any organ, tissue, or cells, as well as urine, or other bodily fluids. The sample can be an tissue biopsy obtained from skin, hair, urine, saliva, semen, feces, sweat, milk, amniotic fluid, liver, heart, muscle, kidney and other body organs. Tissue samples are typically lysed to release the protein and/or nucleic acid content of cells within the samples. The protein or nucleic acid fraction from such crude lysates can then be subject to partial or complete purification before analysis.

Examples of these secreted biomarkers that are suitable for diagnosing cancers are set forth in Table 1 below. For instance, some methods for diagnosing the existence of, or a predisposition to develop, prostate cancer can comprise detecting differentially expressed levels of relaxin-1, MIC-1, or neuropeptide Y. Similarly, detection of differentially expressed MUC-2 may lead to diagnosis of colon cancer. Other than diagnosing cancer in a specific tissue, some methods of the invention are directed to diagnosing cancers in several tissues. For example, detection of a differentially expressed level of mapsin or MUC-1 can indicate the existence of, or a predisposition to develop, a cancer in the prostate, colon, or other tissues (see Examples below). The methods can further comprise examining a subject with a conventional procedure for detecting and diagnosing cancers. Such procedures are well known and routinely practiced in the art, e.g., CAT scanning, MRI, and ultrasonography. Other procedures for diagnosing various forms of cancers are also described in the art, e.g., at http://www.bccancer.bc ca/PPI/TypesofCancer/CancerinGeneral/DiagnosingCancer.

Methods of the present invention are suitable for large scale screening of a population of subjects for the presence or a predisposition to the development of the various forms of cancers. Optionally, the methods can be employed in conjunction with additional biochemical and/or genetic markers of other disorders that may reside in the subjects.

In one aspect of the invention, kits are provided for detecting the level of expression of at least one biomarker identified using the methods of the invention. For example, the kit can comprise a labeled compound or agent capable of detecting a protein encoded by, or mRNA corresponding to, at least one of the biomarkers, means for determining the amount of protein encoded by or mRNA corresponding to the gene or fragment of the protein; and means for comparing the amount of protein encoded by or mRNA corresponding to the gene or fragment of the protein, obtained from the subject sample with a standard level of expression of the gene, e.g., from a cancer-free subject. The compound or agent can be packaged in a suitable container. The kit can further comprise instructions for using the kit to detect protein encoded by or mRNA corresponding to the gene.

The invention also provides methods that are suitable for monitoring subjects who have previously been diagnosed with a cancer, particularly their response to treatment. In another aspect, progression of a cancer in a subject can be monitored by measuring a level of expression of a biomarker identified using the methods of the invention, in a sample of bodily fluid or other tissue obtained in the subject over time, i.e., at various stages of the cancer. An increase in the level of expression of the mRNA or encoded protein corresponding to the gene(s) over time is indicative of the progression of the disorder (e.g., prostate cancer or colon cancer). The level of expression of mRNA and protein corresponding to the gene(s) can be detected by standard methods as described above.

### EXAMPLES

The following examples are provided to illustrate, but not to limit the present invention.

### Example 1

### Identification of Genes Encoding Secreted Proteins

This Example describes an example of the use of the invention to identify biomarkers for cancer. The expression of ˜12,500 transcripts was surveyed in a series of 45 normal and 150 malignant tissue samples representing carcinomas of the prostate, breast, lung, ovary, colorectum, kidney, liver, pancreas, bladder/ureter and stomach/esophagus. A combination of database annotations and predicted amino acid sequence analysis identified a subset of 576 genes that predominantly encode secreted proteins, of which 32 exhibited cancer-specific overexpression. Several of the identified genes encode known or candidate diagnostic proteins, such as mammaglobin in breast cancer, and kallikreins 6 and 10 in ovarian cancer, respectively. We further validated correspondingly high levels of encoded proteins in several cases by immuno-histochemistry on tissue microarrays, or by Western blot analysis of tumor cell conditioned media. The current study demonstrates the combined power of transcript profiling, annotation/protein sequence analysis and immunoassay for the systematic discovery of candidate tumor biomarkers, and highlights several proteins whose detection may improve the sensitivity of cancer diagnosis.

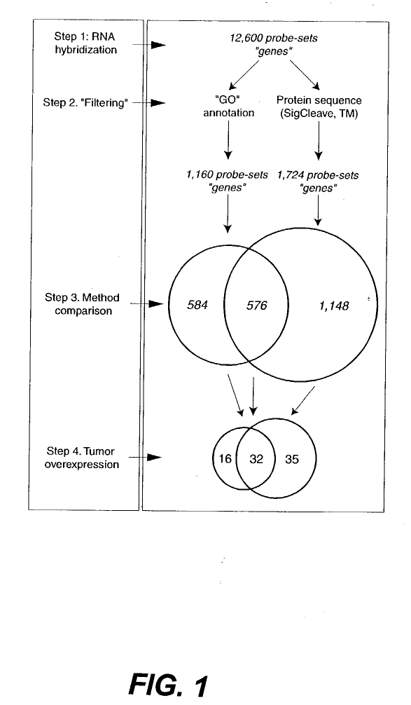

Oligonucleotide probe-sets were filtered for candidate genes encoding secreted proteins by two distinct approaches, as shown in FIG. 1. As shown in FIG. 1A, probe-sets were mapped to Genome Ontology (GO) Consortium annotations (www.genomeontology.org), and those with “location” annotations suggesting protein secretion were identified (1,160). As shown in FIG. 1B, protein sequences of the genes represented on the oligonucleotide microarray were interrogated using two sequence-based algorithms, “tmap” (Persson et al., J Mol Biol, 237:182-192, 1994) and “sigcleave” (von Heijne et al., Nucl. Acids Res., 14:4683-4690, 1986). Sigcleave estimates the likelihood of an authentic signal peptide cleavage site in arbitrary amino acid sequence data; Tmap predicts transmembrane regions in proteins. A series of 1,724 probe-sets (“genes”) met the criteria imposed by both sequence algorithms.

Two approaches were used to select for genes on the Affymetrix U95a GeneChip array that were likely to encode secreted proteins (FIG. 1). First, we asked whether annotation(s) associated with each gene implied secretion of the encoded protein into the extracellular space. Specifically, we mapped probe-sets from the Affymetrix U95a GeneChip to annotations provided by the GO consortium via NCBI's LocusLink database (http://www.ncbi.nlm.nih.gov/LocusLink/), which provides a controlled vocabulary related to a protein's molecular function, biological process, and cellular component. Of the annotations associated with genes on the U95a GeneChip, 1,160 genes were identified by focusing on 30 of the terms. The 30 terms are blood coagulation; blood coagulation factor; cell-cell signaling; cell communication; complement activation; complement component; diuretic hormone; ephrin; extracellular; extracellular matrix; extracellular matrix glycoprotein; extracellular matrix structural protein; extracellular space; hormone; insulin-like growth factor receptor ligand; interleukin 12 receptor ligand; interleukin 2 receptor ligand; interleukin 4 receptor ligand; interleukin 5 receptor ligand; interleukin 6 receptor ligand; interleukin 7 receptor ligand; interleukin 8 receptor ligand; leukemia inhibitor factor receptor ligand; ligand; neuropeptide hormone; opsonin; protein secretion; secreted phospholipase A2; tissue kallikrein; vascular endothelial growth factor receptor ligand.

A parallel approach aimed at genes whose protein sequence features implied the presence of a signal peptide cleavage site, as well as the absence of transmembrane domains, thus suggesting a protein product that would be secreted through a membrane. Conservative thresholds for TMAP and SIGCLEAVE programs (see, e.g., Persson et al., J Protein Chem 16,453-7, 1997; Milpetz et al., Trends Biochem Sci 20, 204-5, 1995; and von Heijne, Nucleic Acids Res 14, 4683-90, 1986) were used for the analyses. With this approach, a set of 1,724 genes were identified that potentially encoded extracellular proteins (FIG. 1).

Together, these two methods identified a subset of 2,308 genes, of which 576 were obtained using both methods.

### Example 2

### Expression of Candidate Genes in Tumors

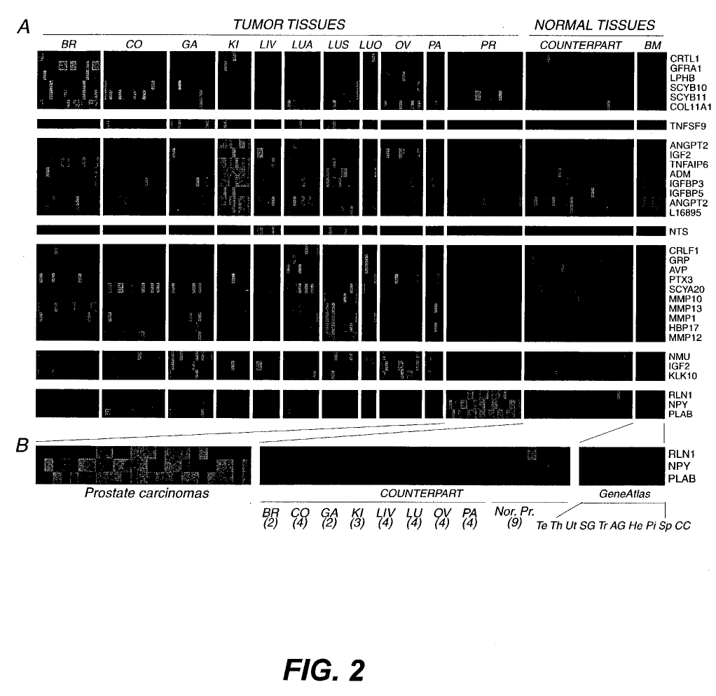

This example describes overexpression of candidate genes encoding secreted proteins in tumors of diverse anatomic origin. Expression of these 2,308 genes was examined in a series of 150 carcinomas representing 10 distinct anatomic origins, 46 normal tissues from the corresponding anatomic sites, and nine other anatomic sites not represented in our “tumor/normal” collection (FIG. 2). What were sought are genes whose expression was high in tumors of one or more sites of origin, with correspondingly low or absent expression in other normal body tissues.

Eighty-three of the 2,308 (3.6%) probe-sets met these criteria (as shown in Table 1), representing 77 different genes. Of the 32 probe-sets (30 different genes) identified by both annotation-and sequence-based analyses (Table 1; FIG. 2A), there was strong evidence that almost all of them encode secreted proteins; only 3/30 genes (the RET co-receptor, GFRA-1; member 9 of the tumor necrosis factor superfamily, TNFR9; and the cytokine receptor-like factor 1, CRLF-1) are unlikely to encode such proteins. For the 16 probe-sets (15 different genes) identified by annotation alone, it was found that 11/15 were secreted. However, only 9/29 unique genes selected by features within their amino acid sequences alone had evidence of secretion. The majority of these genes were found to be GPI-anchored, or integral membrane proteins.

In one study, the mRNAs that correspond to the nucleotide sequences identified using these algorithms as corresponding to mRNAs that encode secreted proteins were analyzed for expression in various cancer and non-cancer samples. As shown in FIGS. 2A-2B, 32 genes encoding secreted proteins were identified with significant over-expression in at least one tumor-normal counterpart tissue pair (>3-fold), and significant over-expression in tumors compared to any other normal tissue (>2-fold).

To confirm the results that shows prostate cancer-specific elevated expression of transcripts for secreted proteins, an RT-PCR analysis was conducted. These results confirmed that the identified genes were indeed overexpressed in prostate cancer cells as compared to non-cancer cells. For further confirmation of these results, an immunoassay was performed in which antibodies specific for candidate biomarker proteins were used to stain tissue microarrays (TMAs) containing 36 epithelial tissues and 229 carcinomas, including those arising in the prostate.

### Example 3

### Validation of Candidate Genes in Tumor Samples

Validation of the above approach first comes from the observation that many of the genes identified here encode secreted proteins previously shown to be dysregulated in cancer tissue (e.g., by other transcript-based approaches, or by IHC), or shown to be elevated in the serum from cancer patients compared to matched controls. The latter include gastrin-releasing peptide (GRP/bombesin) in lung carcinomas (Heasley, Oncogene 20, 1563-9, 2001), kallikreins 6 and 10 (KLK6, KLK10) in ovarian carcinomas (Diamandis et al., Clin Biochem 33, 579-83, 2000; and Luo et al., Clin Chim Acta 306, 111-8, 2001), alpha-fetoprotein (AFP) in liver carcinomas (Johnson et al., Clin Liver Dis 5, 145-59, 2001), and mammaglobin A (MGBA) in breast carcinomas (Fleming et al., Ann N Y Acad Sci 923, 78-89, 2000).

TABLE 1
Genes encoding candidate secreted proteins overexpressed in carcinomas

| Gene Name | Refseq | Evidence | Carcinoma | T/CN | T/ON |
|---|---|---|---|---|---|
| cartilage linking protein 1 | NM_001884 | GO and TMSC | Breast, ER− | 7.0 | 2.9 |
| GDNF family receptor alpha 1 | NM_005264 | GO and TMSC | Breast, ER+ | 9.3 | 9.0 |
| Lipophilin B | NM_006551 | GO and TMSC | Breast, ER+/− | 47.5 | 89.7 |
| small inducible cytokine subfamily B10 | NM_001565 | GO and TMSC | Breast, ER+/− | 10.3 | 6.9 |
| small inducible cytokine subfamily B11 | NM_005409 | GO and TMSC | Breast, ER+/− | 8.1 | 3.7 |
| collagen, type XI, alpha 1 | NM_001854 | GO and TMSC | Breast, ER+/− | 57.3 | 2.7 |
| tumor necrosis factor (ligand) superfamily 9 | NM_003811 | GO and TMSC | Gastric | 3.6 | 3.6 |
| angiopoietin 2 | NM_001147 | GO and TMSC | Kidney | 15.3 | 13.3 |
| insulin-like growth factor 2 (somatomedin A) | NM_000612 | GO and TMSC | Kidney | 50.9 | 8.3 |
| TNF, alpha-induced protein 6 | NM_007115 | GO and TMSC | Kidney | 10.3 | 4.5 |
| Adrenomedullin | NM_001124 | GO and TMSC | Kidney | 3.4 | 3.4 |
| insulin-like growth factor binding protein 3 | NM_000598 | GO and TMSC | Kidney | 6.5 | 2.7 |
| insulin-like growth factor binding protein 5 | NM_000599 | GO and TMSC | Kidney | 4.1 | 2.5 |
| angiopoietin 2 | NM_001147 | GO and TMSC | Kidney | 3.5 | 2.5 |
| lysyl oxidase | NM_002317 | GO and TMSC | Kidney | 3.0 | 2.4 |
| neurotensin | NM_006183 | GO and TMSC | Liver | 96.3 | 9.0 |
| cytokine receptor-like factor 1 | NM_004750 | GO and TMSC | Lung, AdCa | 16.2 | 8.5 |
| gastrin-releasing peptide | NM_002091 | GO and TMSC | Lung, other | 16.5 | 10.4 |
| arginine vasopressin | NM_000490 | GO and TMSC | Lung, other | 4.5 | 5.3 |
| pentaxin-related gene | NM_002852 | GO and TMSC | Lung, other | 15.1 | 4.2 |
| small inducible cytokine subfamily A20 | NM_004591 | GO and TMSC | Lung, other | 5.6 | 3.0 |
| matrix metalloproteinase 10 | NM_002425 | GO and TMSC | Lung, SCC | 15.1 | 18.0 |
| matrix metalloproteinase 13 | NM_002427 | GO and TMSC | Lung, SCC | 7.4 | 7.4 |
| matrix metalloproteinase 1 | NM_002421 | GO and TMSC | Lung, SCC | 24.0 | 5.9 |
| heparmn-binding growth factor binding | NM_005130 | GO and TMSC | Lung, SCC | 24.5 | 2.7 |
| matrix metalloproteinase 12 | NM_002426 | GO and TMSC | Lung, SCC | 32.2 | 2.1 |
| neuromedin U | NM_006681 | GO and TMSC | Ovary | 12.1 | 5.3 |
| kallikrein 10 | NM_002776 | GO and TMSC | Ovary | 5.1 | 2.4 |
| insulin-like growth factor 2 | NM_000612 | GO and TMSC | Ovary | 28.5 | 3.2 |
| (somatomedin A) | | | | | |
| relaxin 1 (HI) | NM_006911 | GO and TMSC | Prostate | 4.6 | 7.8 |
| neuropeptide Y | NM_000905 | GO and TMSC | Prostate | 4.7 | 5.2 |
| MIC-1 | NM_004864 | GO and TMSC | Prostate | 6.7 | 3.5 |
| mucin 2, intestinal/tracheal | NM_002457 | GO only | Colon | 8.8 | 5.3 |
| interleukin 18 | NM_001562 | GO only | Gastric | 3.8 | 2.6 |
| Indian hedgehog homolog (Drosophila) | none | GO only | Gastric | 4.7 | 2.4 |
| adipose differentiation-related protein | NM_001122 | GO only | Kidney | 9.6 | 2.6 |
| vascular endothelial growth factor receptor | NM_002019 | GO only | Kidney | 3.8 | 2.4 |
| alpha-fetoprotein | NM_001134 | GO only | Liver | 7.6 | 7.6 |
| hypocretin (orexin) receptor 1 | NM_001525 | GO only | Liver | 5.4 | 5.4 |
| interleukin 1, beta | NM_000576 | GO only | Lung, other | 33.5 | 33.5 |
| neurotensin receptor 1 (high affinity) | NM_002531 | GO only | Long, other | 10.3 | 10.3 |
| plasminogen activator inhibitor type 1 | NM_000602 | GO only | Lung, other | 43.4 | 5.4 |
| ephrin-B2 | NM_004093 | GO only | Lung, other | 3.5 | 2.8 |
| interleukin 1, beta | NM_000576 | GO only | Lung, other | 3.1 | 2.2 |
| epiregulin | NM_001432 | GO only | Lung, other | 4.2 | 2.1 |
| Galectin 7 | NM_002307 | GO only | Lung, SCC | 39.7 | 34.6 |
| plakophilin 1 | NM_000299 | GO only | Lung, SCC | 7.9 | 7.9 |
| WNT7A | NM_004625 | GO only | Ovary | 6.2 | 6.2 |
| E2F transcription factor 3 | NM_001949 | TMSC Only | Breast, ER- | 10.8 | 6.6 |
| FLJ20244 | Hs.351792 | TMSC Only | Breast, ER- | 3.7 | 3.7 |
| carbohydrate sulfotransferase 2 | NM_004267 | TMSC Only | Breast, ER- | 7.9 | 2.0 |
| serine hydrolase-like | NM_014509 | TMSC Only | Breast | 17.5 | 17.5 |
| FLJ13927 | Hs.343963 | TMSC Only | Breast | 6.6 | 11.6 |
| mammaglobin A | NM_002411 | TMSC Only | Breast | 9.0 | 8.9 |
| cytochrome c oxidase subunit Vic | NM_004374 | TMSC Only | Breast | 3.5 | 2.9 |
| mammaglobin B | NM_002407 | TMSC Only | Breast | 14.2 | 2.6 |
| defensin, alpha 6, Paneth cell-specific | NM_001926 | TMSC Only | Gastric | 20.5 | 5.7 |
| proline 4-hydroxylase | NM_000918 | TMSC Only | Gastric | 4.3 | 4.3 |
| defensin, alpha 5, Paneth cell-specific | NM_021010 | TMSC Only | Gastric | 3.8 | 3.9 |
| endothelial cell-specific molecule 1 | NM_007036 | TMSC Only | Kidney | 6.6 | 6.5 |
| cerebroside sulfotransferase | NM_004861 | TMSC Only | Kidney | 9.9 | 6.3 |
| vanin 1 | NM_004666 | TMSC Only | Kidney | 4.9 | 4.9 |
| TNF-r superfamily, member 17 | NM_001192 | TMSC Only | Liver | 4.2 | 3.1 |
| bone morphogenetic protein 6 | NM_001718 | TMSC Only | Lung, AdCa | 3.1 | 3.1 |
| Zic family member 3 heterotaxy 1 | NM_003413 | TMSC Only | Lung, other | 6.5 | 6.5 |
| achaete-scute complex-like 1 (Drosophila) | NM_004316 | TMSC Only | Lung, otber | 55.2 | 5.7 |
| achacte-scute complex-like 1 (Drosophila) | NM_004316 | TMSC Only | Lung, other | 28.9 | 5.1 |
| ovalbumin | NM_002640 | TMSC Only | Lung, other | 4.6 | 4.6 |
| TSS candidate 3 | NM_003311 | TMSC Only | Lung, other | 10.8 | 4.3 |
| TSS candidate 3 | NM_003311 | TMSC Only | Lung, other | 7.1 | 2.4 |
| transcription factor A, mitochondrial | NM_003201 | TMSC Only | Lung, other | 3.4 | 2.1 |
| lymphocyte antigen 6 complex, locus D | NM_003695 | TMSC Only | Lung, SCC | 50.7 | 14.5 |
| ovalbumin | NM_002639 | TMSC Only | Lung, SCC | 68.1 | 5.5 |
| ovalbumin | NM_002639 | TMSC Only | Lung, SCC | 29.3 | 3.4 |
| GPI-anchored metastasis protein | NM_014400 | TMSC Only | Lung, SCC | 5.0 | 2.5 |
| melanoma antigen, family A, 5 | NM_021049 | TMSC Only | Lung, SCC | 4.8 | 2.2 |
| kallikrein 6 (neurosin, zyme) | NM_002774 | TMSC Only | Ovary | 15.2 | 2.1 |
| mesothelin | NM_005823 | TMSC Only | Ovary | 23.0 | 2.1 |
| pancreatic thread protein-like (rat) | NM_006508 | TMSC Only | Pancreas | 3.3 | 13.3 |
| prostate-specific membrane antigen | NM_004476 | TMSC Only | Prostate | 3.9 | 4.9 |
| prostate-specific membrane antigen | Hs.283946 | TMSC Only | Prostate | 4.2 | 4.2 |
| prostate-specific membrane antigen | NM_004476 | TMSC Only | Prostate | 3.9 | 3.6 |
| single-minded homolog 2 (Drosophila) | NM_005069 | TMSC Only | Prostate | 10.6 | 2.7 |

Table notes:
T/CN = ratio of expression of tumor tissue and corresponding normal tissue
T/OtherN = ratio of expression of tumor tissue and “other” normal tissues
Evidence = whether the genes were identified by GO annotations and sequence analysis (GO and TMSC); GO annotations alone (GO Only) or sequence analysis alone (TMSC Only)
Breast, ER+ = estrogen receptor positive breast carcinoma
Breast, ER− = estrogen receptor negative breast carcinoma
Breast, ER+/− = breast carcinoma with undetermined ER status
Lung, AdCa = lung adeocarcinoma
Lung, SCC = lung squamous cell carcinoma
Lung, other = lung carcinomas with histologies other than squamous or adeocarcinoma

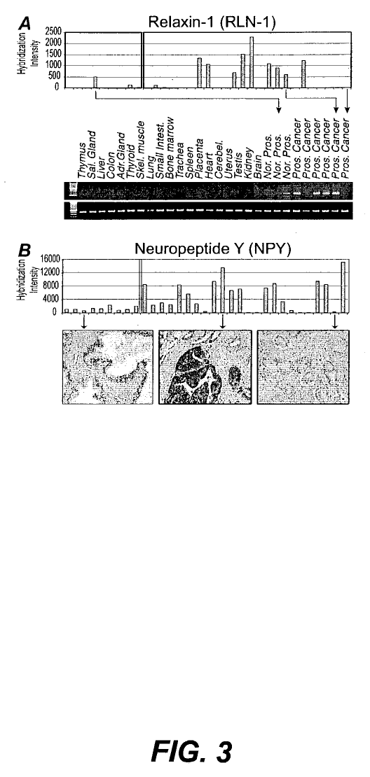

In addition, for many of the other genes discovered using this approach, tumor overexpression was validated using several independent methods. For example, in prostate carcinomas (highlighted in FIG. 2B), the highly tissue-selective expression of relaxin-1 (RLN1), a small peptide hormone of the insulin family involved in remodeling the birth canal (Bani, Gen Pharmacol 28, 13-22, 1997), was confirmed by semi-quantitative RT-PCR in nine prostate and 19 non-prostatic tissues. Expression levels of RLN-1 determined by PCR were entirely consistent with the results obtained by microarray hybridization (FIG. 3A). These results confirmed that the identified genes were indeed overexpressed in prostate cancer cells as compared to non-cancer cells.

For neuropeptide Y (NPY), which was highly expressed in 15/25 prostate carcinomas compared to normal prostate tissue, tissue microarrays containing 229 carcinomas and 36 normal tissues samples of diverse anatomic origin were stained with a commercial anti-NPY antibody. In normal prostate tissues, staining was found in nerves and a few prostate secretory epithelial cells, while in prostate carcinomas that had high NPY gene expression, correspondingly high levels of protein expression were found (FIG. 3B). The anti-NPY antibody did not stain carcinomas of other anatomic sites on the TMAs, nor other non-neural or non-neuroendocine normal tissues that were included on these arrays.

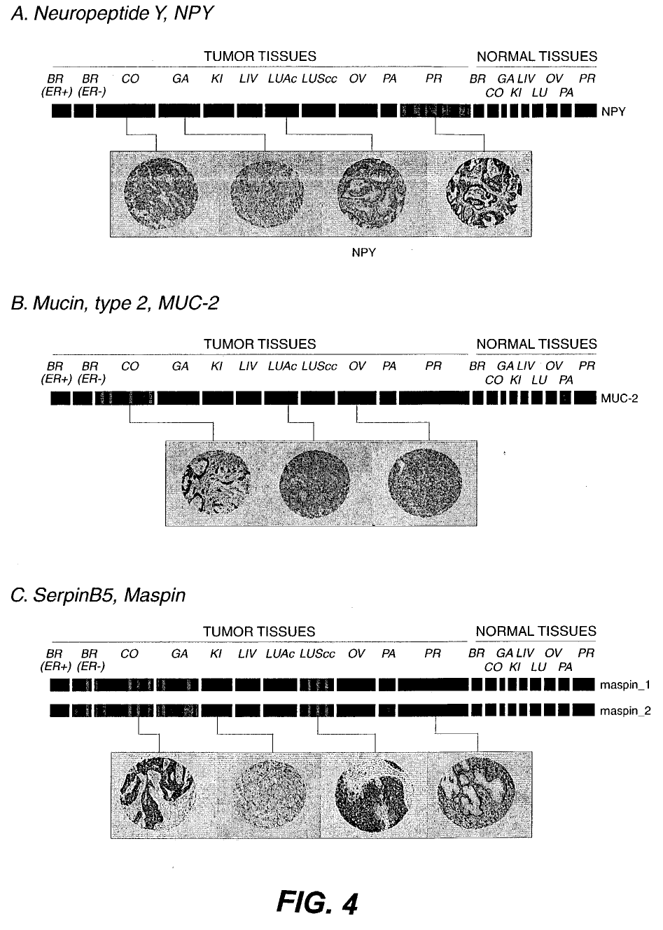

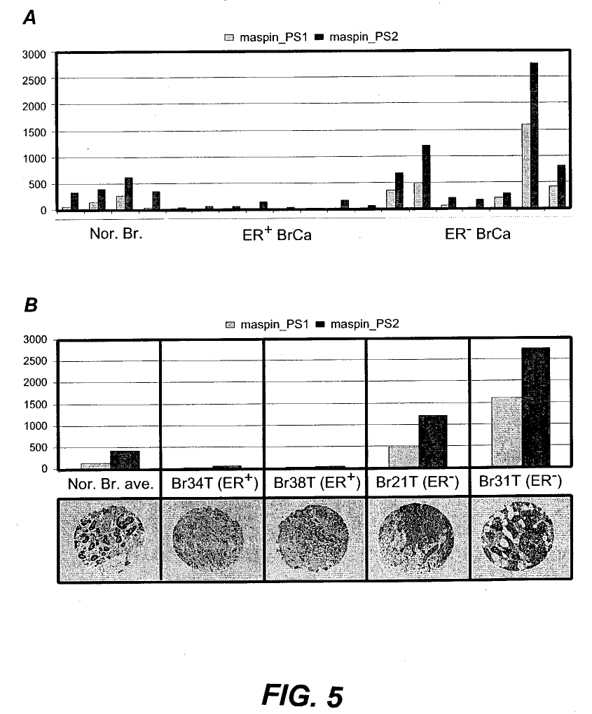

In carcinomas of other sites, similarly consistent results were also obtained using antibodies directed against other candidate proteins. As expected from transcript analysis, antibodies specific for MUC-2 showed selective expression in carcinomas of the colon, whereas antibodies specific for maspin, showed high expression in carcinomas of the colon, gastroesophagous, lung and pancreas compared to normal tissue sites (FIGS. 4A-4C). Decreased maspin expression was found in some ductal carcinomas of the breast as compared to normal ductal breast tissue, consistent with its purported role as a tumor suppressor in breast cancer (Sager et al., Adv Exp Med Biol 425, 77-88, 1997). However, cases of breast carcinoma with significantly elevated levels of the maspin protein were also observed (FIGS. 5A-5B). In a small series of breast tumors, it was found that maspin expression correlated with estrogen receptor status of the tumor, consistent with recent reports of maspin gene overexpression in ER-negative carcinomas (Martin et al., Cancer Res 60, 2232-8, 2000).

### Example 4

### Overexpression of Candidate Genes in Independent Datasets

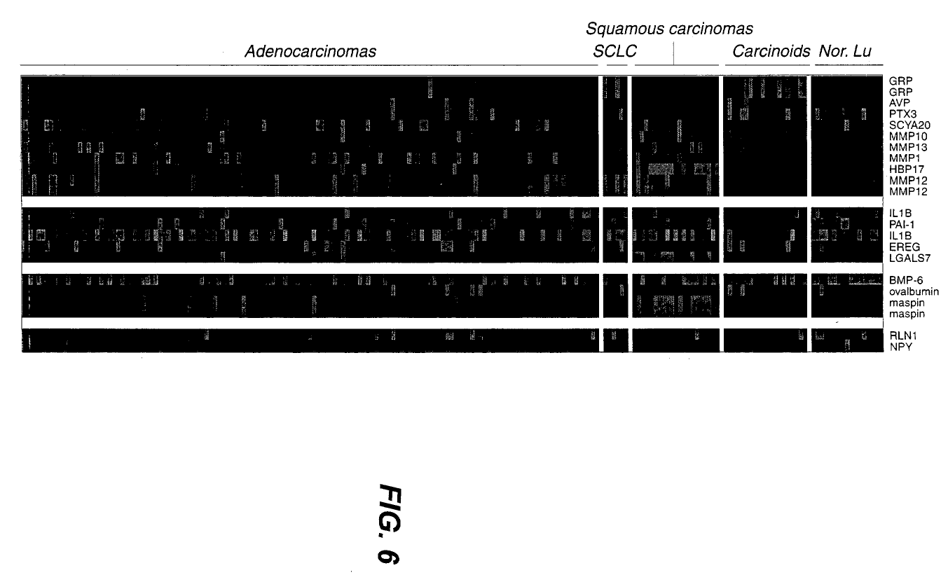

The instant example describes candidate gene expression in other independent datasets. For example, publicly accessible data from Bhattacharjee et al. (Proc Natl Acad Sci U S A 98, 13790-5, 2001) enabled analysis of expression of the lung cancer candidate genes of the present invention in 203 lung tissues, including 17 samples of normal lung, 127 adenocarcinomas, 21 squamous carcinomas, 20 pulmonary carcinoids, and 6 small cell undifferentiated carcinomas. This analysis demonstrated some striking correlations of gene expression with histological subtype. For example, GRP/bombesin was overexpressed predominantly in small cell lung carcinomas and carcinoids, consistent with published reports (Sunday et al., Pathol 22, 1030-9, 1991), and in only 2/127 adenocarcinomas; maspin, in contrast was expressed in almost all of the squamous cell carcinomas, but only in a minority of adenocarcinomas, and not at all in carcinoids. The specificity of these results is reflected by the relative lack of expression of RLN1 and NPY, which were included as controls based on their predominantly prostate cancer-specific expression (FIG. 6). Compared to normal lung tissue, and based on their relative histological specificity, it appears that proteins such as maspin, as well as heparin-binding protein 17 (HBP-17) and galectin 7, are useful diagnostic biomarkers.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A method for identifying a biomarker that is diagnostic for the presence of a cancer in a mammal, the method comprising:
analyzing one or more polynucleotide sequences using an algorithm that determines whether the polynucleotide sequence is predicted to encode a polypeptide that is secreted from a cell in which the polypeptide is expressed; and
determining whether an mRNA that corresponds to one or more of the polynucleotide sequences that are predicted to encode secreted polypeptides is differentially expressed in one or more types of cancer cells compared to non-cancer cells;
wherein an mRNA that is differentially expressed in cancer cells compared to non-cancer cells, or a polypeptide encoded by the differentially expressed mRNA, is useful as a biomarker that is diagnostic for the presence of the cancer in a mammal.

2. The method of claim 1, wherein the method is performed on a plurality of polynucleotide sequences that are present in a database.

3. The method of claim 2, wherein the database comprises 1,000 or more polynucleotide sequences.

4. The method of claim 3, wherein the database comprises 10,000 or more polynucleotide sequences.

5. The method of claim 2, wherein the database is provided by the Gene Ontology Consortium.

6. The method of claim 1, wherein the algorithm comprises identifying polynucleotide sequences for which an associated annotation indicates that a polypeptide encoded by the polynucleotide sequence is secreted from a cell.

7. The method of claim 1, wherein the algorithm comprises identifying polynucleotide sequences that encode a predicted amino acid sequence that comprises a transmembrane domain.

8. The method of claim 7, wherein the algorithm comprises Tmap.

9. The method of claim 1, wherein the algorithm comprises identifying polynucleotide sequences that encode a predicted amino acid sequence that comprises one or more of a signal polypeptide and a signal polypeptide cleavage site.

10. The method of claim 9, wherein the algorithm comprises SigCleave.

11. The method of claim 1, wherein two or more algorithms are used that identify polynucleotide sequences that are predicted to encode polypeptides that are secreted from a cell.

12. The method of claim 1, wherein the polynucleotide sequence or sequences are analyzed by identifying associated annotations that are indicative of secretion and by one or both of Tmap and SigCleave.

13. The method of claim 1, wherein the determination of differential expression is performed using a polynucleotide array that comprises a plurality of probes to which can hybridize an mRNA, cRNA, or cDNA that is present in a sample obtained from the cancer cells or non-cancer cells.

14. The method of claim 13, wherein the polynucleotide array comprises a GeneChip@.

15. The method of claim 1, wherein differential expression is assessed in cells obtained from a plurality of different cancers.

16. The method of claim 15, wherein differential expression is assessed in a cells obtained from a plurality of samples for each of the different cancers.

17. The method of claim 1, wherein the cancer cells are obtained from a cancer selected from the group consisting of prostate, breast, lung, ovary, colorectum, kidney, liver, pancreas, bladder/ureter and stomach/esophagus cancer.

18. A method of diagnosing a cancer in a mammal, the method comprising detecting in a sample obtained from the mammal an increase in the level of a biomarker, wherein the biomarker was identified using a method that comprises:
analyzing one or more polynucleotide sequences using an algorithm that determines whether the polynucleotide sequence is predicted to encode a polypeptide that is secreted from a cell in which the polypeptide is expressed; and
determining whether an mRNA that corresponds to one or more of the polynucleotide sequences that are predicted to encode secreted polypeptides is differentially expressed in one or more types of cancer cells compared to non-cancer cells;
wherein an mRNA that is differentially expressed in cancer cells compared to non-cancer cells, or a polypeptide encoded by the differentially expressed mRNA, is useful as a biomarker that is diagnostic for the presence of the cancer in a mammal.

19. The method of claim 18, wherein the cancer cells are obtained from a cancer selected from the group consisting of prostate, breast, lung, ovary, colorectum, kidney, liver, pancreas, bladder/ureter and stomach/esophagus cancer.

20. The method of claim 18, wherein the biomarker is a polypeptide encoded by the differentially expressed mRNA.

21. The method of claim 19, wherein the sample comprises blood or serum obtained from the mammal.

22. The method of claim 18, wherein the mammal is a human.

23. A method for monitoring the efficacy of a cancer treatment in a mammal, the method comprising detecting an increase or decrease in the level of a biomarker that is diagnostic for the presence of the cancer in a mammal in a plurality of samples obtained from the mammal at different times, wherein the biomarker was identified using a method that comprises:
analyzing one or more polynucleotide sequences using an algorithm that determines whether the polynucleotide sequence is predicted to encode a polypeptide that is secreted from a cell in which the polypeptide is expressed; and
determining whether an mRNA that corresponds to one or more of the polynucleotide sequences that are predicted to encode secreted polypeptides is differentially expressed in one or more types of cancer cells compared to non-cancer cells;
wherein an mRNA that is differentially expressed in cancer cells compared to non-cancer cells, or a polypeptide encoded by the differentially expressed mRNA, is useful as a biomarker that is diagnostic for the presence of the cancer in a mammal.
The method of claim 23, wherein the mammal was subjected to a cancer treatment in between obtaining two or more of the samples.

24. The method of claim 23, wherein the mammal was subjected to a cancer treatment in between obtaining two or more of the samples.

25. A method for diagnosing, or identifying a predisposition to the development of, prostate cancer in a mammal, comprising (a) obtaining a biological sample from the mammal suspected to have or be predisposed to develop prostate cancer; and (b) detecting in the biological sample an abnormal level of at least one secreted biomarker of prostate cancer, thereby diagnosing or identifying a predisposition to the development of prostate cancer in the mammal; wherein the at least one secreted biomarker of prostate cancer is selected from the group consisting of relaxin 1 (H1), neuropeptide Y, MIC-1, pancreatic thread protein-like (rat), prostate-specific membrane antigen, prostate-specific membrane antigen, prostate-specific membrane antigen, and single-minded homolog 2 (Drosophila).

26. The method of claim 25, wherein the biological sample is serum, blood plasma, or whole blood.

27. The method of claim 25, wherein the mammal has elevated expression level of said biomarker than level of the biomarker in a control population unaffected by cancer.

28. The method of claim 25, furthering comprising examining the mammal with a conventional procedure for diagnosing cancer.

29. A method for diagnosing, or identifying a predisposition to the development of, colon cancer in a mammal, comprising (a) obtaining a biological sample from the mammal suspected to have or be predisposed to develop colon cancer, and (b) detecting in the biological sample an elevated level of mucin 2 (MUC-2).

30. A method for diagnosing, or identifying a predisposition to the development of, a carcinoma in a mammal, comprising (a) obtaining a biological sample from the mammal suspected to have or be predisposed to develop a carcinoma, and (b) detecting in the biological sample from the mammal an elevated level of mapsin; wherein said carcinoma is colon cancer, gastroesophagous cancer, lung cancer, or pancreas cancer.
